(19) United States
(12) Patent Application Publication
(10) Pub. No.: US 2026/0269025 A1
(43) Pub. Date: Sep. 10, 2026

(54) METHOD FOR CALCULATING RECYCLED MATERIAL RATIO IN POLYMER MATERIAL BASED ON ACCELERATED THERMAL OXIDATIVE AGING

(71) Applicants: Hyundai Motor Company, Seoul (KR); Kia Corporation, Seoul (KR)

(72) Inventors: Yurong Gao, Yantai (CN); Hong Mo Koo, Hwaseong-si (KR)

(73) Assignees: Hyundai Motor Company, Seoul (KR); Kia Corporation, Seoul (KR)

(21) Appl. No.: 19/248,236

(22) Filed: Jun. 24, 2025

(30) Foreign Application Priority Data
Mar. 4, 2025 (CN) 202510248444.5

(51) Int. Cl. G16C 60/00; G16C 20/30

(52) U.S. Cl. CPC G16C 60/00; G16C 20/30

(57) ABSTRACT

The present disclosure provides a method for calculating a recycled material ratio in a polymer material based on accelerated thermal oxidative aging, the method including preparing a plurality of standard samples, performing thermal oxidative aging, and measuring a characteristic property, fitting an aging curve according to data of the characteristic property, and obtaining a peak degradation rate (R) and/or degradation time (T90−70), according to the peak degradation rate (R) and/or the degradation time (T90−70), and the recycled material ratio, obtaining a relationship between the peak degradation rate (R) and/or the degradation time (T90−70) and a recycled material ratio through fitting, preparing a sample for analysis, performing thermal oxidative aging, and measuring the characteristic property, fitting an aging curve according to the data of the characteristic property, and obtaining the peak degradation rate (R) and/or degradation time (T90−70) of the sample for analysis, and calculating the recycled material ratio of the sample for analysis according to the obtained relationship and the obtained peak degradation rate (R) and/or degradation time (T90−70) of the sample for analysis.

### CROSS-REFERENCE TO RELATED APPLICATION

This present application claims the benefit of priority to Chinese Patent Application No. 202510248444.5, filed on Mar. 4, 2025, in the China National Intellectual Property Administration, the entire disclosure of which is incorporated herein by reference in its entirety.

### FIELD

The present disclosure relates to a method for calculating a recycled material ratio in a polymer material based on accelerated thermal oxidative aging, and a method for determining whether a polymer material contains recycled material based on accelerated thermal oxidative aging.

### BACKGROUND

In 2023, the European Commission proposed mandating that all newly manufactured automobiles incorporate a minimum of 25% recycled materials. This initiative was part of a broader strategy aimed at promoting sustainability within the automotive industry. Nonetheless, original equipment manufacturers (OEMs) face significant challenges in identifying and calculating the ratio of recycled materials utilized in their products. The determination of these ratios necessitates sophisticated methodologies and accurate tracking procedures, which have not yet been fully standardized across the industry.

To monitor the recycled material ratios effectively, OEMs currently rely on obtaining certification, a process that incurs substantial costs due to the stringent requirements set forth by regulatory bodies.

In response to these challenges, several research institutions worldwide are attempting to develop methods for the identification and quantification of recycled material ratios in polymer-based substances. Thus, there is a need for improved compositions and methods that facilitate better management of the supply chain for recycled materials within the automotive sector, as well as promote the responsible sourcing and utilization of recycled materials, thereby driving industry-wide sustainability initiatives.

### SUMMARY

Typically, thermal oxidation resistance of polymer materials is evaluated through the degradation onset time, and according to extensive research results, polymer materials containing recycled material begin to degrade faster than virgin materials under thermal oxidative aging conditions. However, it is difficult to distinguish between recycled materials and virgin materials because antioxidants can be added to effectively delay the degradation onset time of materials to match that of virgin materials. Therefore, a method is needed to identify the recycled material ratio in polymer materials obtained from material suppliers.

The present disclosure is directed to providing a method capable of performing quantitative analysis of a recycled material ratio in a polymer material while excluding the influence of antioxidants, and a method capable of simply determining whether a polymer material contains recycled material.

The inventors of the present disclosure discovered that, when excluding the interference of antioxidants (ensuring sufficient amounts of primary antioxidants and secondary antioxidants), during the overall thermal oxidative aging degradation process, polymer materials containing different ratios of recycled material exhibit different peak degradation rates (sometimes also abbreviated as “degradation rate”) (R) and degradation times (T90−70) (the degradation time from P/P0=0.9 to P/P0=0.7 (T90−70) (where P/P0 represents the ratio of the value of a specified characteristic property during the thermal oxidative aging process to the value of that characteristic property before the start of thermal oxidative aging)).

Additionally, the inventors of the present disclosure discovered through thermal oxidative aging of various materials that if the peak degradation rate (R) is less than a specified peak degradation rate and the degradation time (T90−70) is greater than a specified degradation time, the material is typically a virgin material (0% recycled material ratio).

Based on these findings, the inventors of the present disclosure have established a method capable of identifying the recycled material ratio in a polymer material by calculating the peak degradation rate and/or degradation time of the polymer material during the thermal oxidative aging process, and a method capable of determining whether a polymer material contains recycled material.

According to an aspect of the present disclosure, a method for calculating a recycled material ratio in a polymer material based on accelerated thermal oxidative aging is provided, the method including: preparing a plurality of standard samples of the polymer material, wherein the plurality of standard samples have a different recycled material ratio and contain primary antioxidants and secondary antioxidants, performing thermal oxidative aging on the plurality of standard samples at a specified temperature, and measuring data of a specified characteristic property of each of the plurality of standard samples at a plurality of times from a start of thermal oxidative aging; fitting an aging curve for each of the plurality of standard samples according to the data of the specified characteristic property measured during the thermal oxidative aging, and obtaining at least one of a peak degradation rate (R) or a degradation time (T90−70) from P/P0=0.9 to P/P0=0.7 from the aging curve, wherein P/P0 represents a ratio of a value of the specified characteristic property during the thermal oxidative aging to a value of the specified characteristic property before the start of the thermal oxidative aging; according to at least one of the peak degradation rate (R) or the degradation time (T90−70) of each of the plurality of standard samples, and the recycled material ratio, obtaining at least one of a relationship between the peak degradation rate (R) and the recycled material ratio or a relationship between the degradation time (T90−70) and the recycled material ratio through fitting; preparing a sample of the polymer material for analysis, and in a state in which the sample for analysis contains primary antioxidants and secondary antioxidants, performing thermal oxidative aging on the sample for analysis under identical conditions to the thermal oxidative aging of the plurality of standard samples, and measuring data of the specified characteristic property; fitting an aging curve for the sample for analysis according to the data of the specified characteristic property measured during the thermal oxidative aging, and obtaining at least one of the peak degradation rate (R) or the degradation time (T90−70) from P/P0=0.9 to P/P0=0.7 of the sample for analysis from the aging curve; and calculating the recycled material ratio in the sample for analysis according to at least one of the obtained relationship between the peak degradation rate (R) and the recycled material ratio, and the obtained peak degradation rate (R) of the sample for analysis, or the obtained relationship between the degradation time (T90−70) and the recycled material ratio, and the obtained degradation time (T90−70) of the sample for analysis.

In some embodiments, the calculating the recycled material ratio in the sample for analysis is included, in a situation in which the recycled material ratio in the sample for analysis is calculated according to the relationship between the peak degradation rate (R) and the recycled material ratio and the recycled material ratio in the sample for analysis is calculated according to the relationship between the degradation time (T90−70) and the recycled material ratio, selecting an average value of the two calculated recycled material ratios as the recycled material ratio in the sample for analysis.

In some embodiments, the fitting an aging curve for each of the plurality of standard samples is included, for each of the plurality of standard samples, arranging the data of the specified characteristic property in chronological order, fitting the aging curve through a modified Weibull survival function, calculating a derivative of the aging curve, and obtaining at least one of the peak degradation rate (R) or the degradation time (T90−70) from the derivative of the aging curve.

In some embodiments, the fitting an aging curve for the sample for analysis is included, for the sample for analysis, arranging the data of the specified characteristic property in chronological order, fitting the aging curve through a modified Weibull survival function, calculating a derivative of the aging curve, and obtaining at least one of the peak degradation rate (R) or the degradation time (T90−70) from the derivative of the aging curve.

In some embodiments, the polymer material is PP-GF30, the specified characteristic property is tensile strength, and the obtained relationship between the peak degradation rate (R) and the recycled material ratio is R=P1+P2*ratio, wherein a unit of the peak degradation rate (R) is (1000 hours)⁻¹, 0.1525≤P1≤0.2831, and 3.941≤P2≤7.319. Here, the recycled material ratio is a number from 0 to 1.0 in calculation, and the calculated recycled material ratio can also be written in the form of a percentage.

In some embodiments, P1=0.2178 and P2=5.630.

In some embodiments, the polymer material is PP-GF30, the specified characteristic property is tensile strength, and the obtained relationship between the degradation time (T90−70) and the recycled material ratio is T90−70=P3+P4*ratio, wherein a unit of the degradation time (T90−70) is hours, 174.8≤P3≤324.6, and −289.1≤P4≤−155.7. Here, the recycled material ratio is a number from 0 to 1.0 in calculation, and the calculated recycled material ratio can also be written in the form of a percentage.

In some embodiments, P3=249.7 and P4=−222.4.

In some embodiments, the polymer material is PP-GF30, the specified characteristic property is TGA degradation onset temperature, and the obtained relationship between the peak degradation rate (R) and the recycled material ratio is R=T1+T2*ratio, wherein a unit of the peak degradation rate (R) is degrees Celsius/hour, −0.00238<T1≤−0.00128, and 0.08722<T2<0.1620. Here, the recycled material ratio is a number from 0 to 1.0 in calculation, and the calculated recycled material ratio can also be written in the form of a percentage.

In some embodiments, T1=−0.00183, and T2=0.1246.

In some embodiments, the preparing a plurality of standard samples includes providing the primary antioxidants in an amount sufficient to perform 1000 hours of thermal oxidative aging under the specified temperature, and providing the secondary antioxidants in an amount sufficient to remain present until an end of thermal oxidative aging.

In some embodiments, the preparing a sample of the polymer material for analysis is included, before performing thermal oxidative aging on the sample for analysis, analyzing a ratio of primary antioxidants and secondary antioxidants in the sample for analysis to ensure that an amount of the secondary antioxidants is sufficient during the thermal oxidative aging, and based on the amount of the secondary antioxidants being insufficient, adding secondary antioxidants until sufficient.

According to another aspect of the present disclosure, a method for determining whether a polymer material contains recycled material based on accelerated thermal oxidative aging is provided, the method including: preparing a sample of a polymer material for analysis, and in a state in which the sample for analysis contains primary antioxidants and secondary antioxidants, performing thermal oxidative aging on the sample for analysis at a specified temperature, and measuring data of a specified characteristic property of the sample for analysis at a plurality of times from a start of thermal oxidative aging; fitting an aging curve for the sample for analysis according to the data of the specified characteristic property measured during the thermal oxidative aging, and obtaining a peak degradation rate (R) and a degradation time (T90−70) from P/P0=0.9 to P/P0=0.7 of the sample for analysis from the aging curve, wherein P/P0 represents a ratio of a value of the specified characteristic property during the thermal oxidative aging to a value of the specified characteristic property before the start of thermal oxidative aging; and determining that the sample for analysis is a virgin material not containing recycled material based on the peak degradation rate (R) being less than a first threshold and the degradation time (T90−70) being greater than a second threshold.

In some embodiments, the determining the sample for analysis includes determining that the sample for analysis contains recycled material based on the peak degradation rate (R) being greater than or equal to the first threshold or the degradation time (T90−70) being less than or equal to the second threshold.

In some embodiments, the fitting an aging curve for the sample for analysis includes arranging the data of the specified characteristic property in chronological order, fitting the aging curve through a modified Weibull survival function, calculating a derivative of the aging curve, and obtaining the peak degradation rate (R) and the degradation time (T90−70) from the derivative of the aging curve.

In some embodiments, the polymer material is PP-GF40 or PP-GF30, the specified characteristic property is tensile strength, the first threshold is 1.0(1000 hours)⁻¹, and the second threshold is 200 hours.

Through the present disclosure, a method capable of performing quantitative analysis of a recycled material ratio in polymer materials while excluding the influence of antioxidants, and a method capable of simply determining whether a polymer material contains recycled material, may be provided.

### BRIEF DESCRIPTION OF THE DRAWINGS

The foregoing and other aspects, features, and advantages, as well as the following detailed description of the embodiments, will be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings indicate like elements.

FIG. 1a is a diagram showing the change of tensile strength retention rate over time and the change of degradation rate over time during a thermal oxidative aging process of four standard samples in one embodiment of the present disclosure.

FIG. 1b is a fitting diagram showing the relationship between the degradation time (T90−70) and the recycled material ratio in this embodiment of the present disclosure.

FIG. 1c is a fitting diagram showing the relationship between the peak degradation rate (R) and the recycled material ratio in this embodiment of the present disclosure.

FIG. 2 is a diagram showing the change of tensile strength retention rate over time and the change of degradation rate over time during the thermal oxidative aging process of two samples for analysis in this embodiment of the present disclosure.

FIG. 3a is a diagram showing the change of tensile strength retention rate over time and the change of degradation rate over time during the thermal oxidative aging process of a virgin material (sample number 7) in another embodiment of the present disclosure.

FIG. 3b is a diagram showing the change of tensile strength retention rate over time and the change of degradation rate over time during the thermal oxidative aging process of another virgin material (sample number 8) in this embodiment of the present disclosure.

FIG. 4a is a diagram showing the change over time of the decrease in TGA thermal decomposition onset temperature compared to the initial sample during the thermal oxidative aging process of three standard samples, and the change of degradation rate over time, in still another embodiment of the present disclosure.

FIG. 4b is a fitting diagram showing the relationship between the peak degradation rate (R) and the recycled material ratio in this embodiment of the present disclosure.

It shall be understood that, for simplification and/or clear illustration, the elements shown in the attached drawings are not necessarily drawn to scale. For example, for clarity, the dimensions of some elements may be exaggerated relative to other elements. In addition, to facilitate understanding of the concepts of the present disclosure, known elements may be omitted from the attached drawings. The dimensions in the attached drawings do not represent the exact dimensions and/or dimensional ratios of each element of this specification.

### DETAILED DESCRIPTION

To make the purpose, technical solutions, and advantages of embodiments of the present disclosure clearer, embodiments will be described in detail below with reference to the accompanying drawings. It is understood that the described embodiments are merely some embodiments of the present disclosure, not all embodiments. Other embodiments obtained by those skilled in the art without creative efforts based on the embodiments of the present disclosure all fall within the scope of protection of the present disclosure.

Unless otherwise defined, all technical and scientific terms used in the present disclosure have the same meanings as commonly understood by those skilled in the art of this application. The terms “include,” “comprise,” and any variations thereof used in the specification of this application are intended to encompass non-exclusive inclusion.

In the description of the present disclosure, it shall be noted that, unless explicitly defined or limited otherwise, terms such as “mounted,” “interconnected,” “connected,” etc., should be interpreted broadly. For example, they may be fixed connections, detachable connections, or integral connections, and they may be directly interconnected, or indirectly interconnected through intermediate media. Those of ordinary skill in the art will understand the specific meanings of the above-mentioned terms in the present application according to the specific situation.

The present disclosure may use ordinal numbers such as “first,” “second,” “third,” etc., to refer to elements, and it should be noted that, unless explicitly indicated otherwise, these are merely used to distinguish between different elements and do not imply that the mentioned elements are necessarily provided in the indicated order in terms of time, space, or other aspects.

### One Embodiment of the Present Disclosure

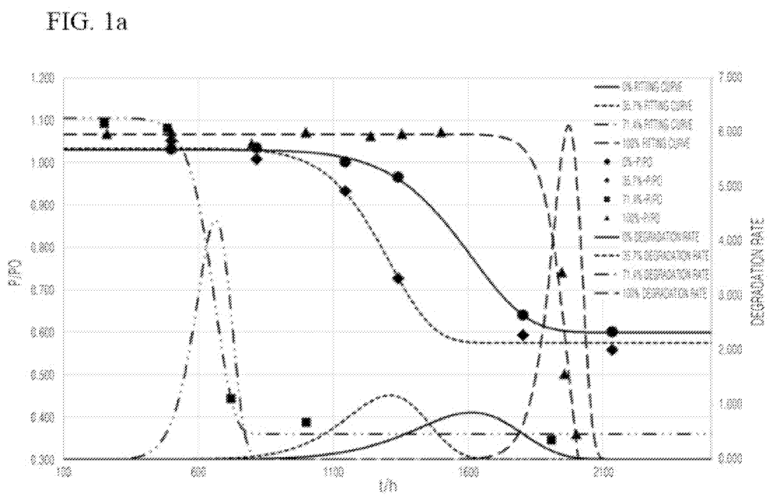

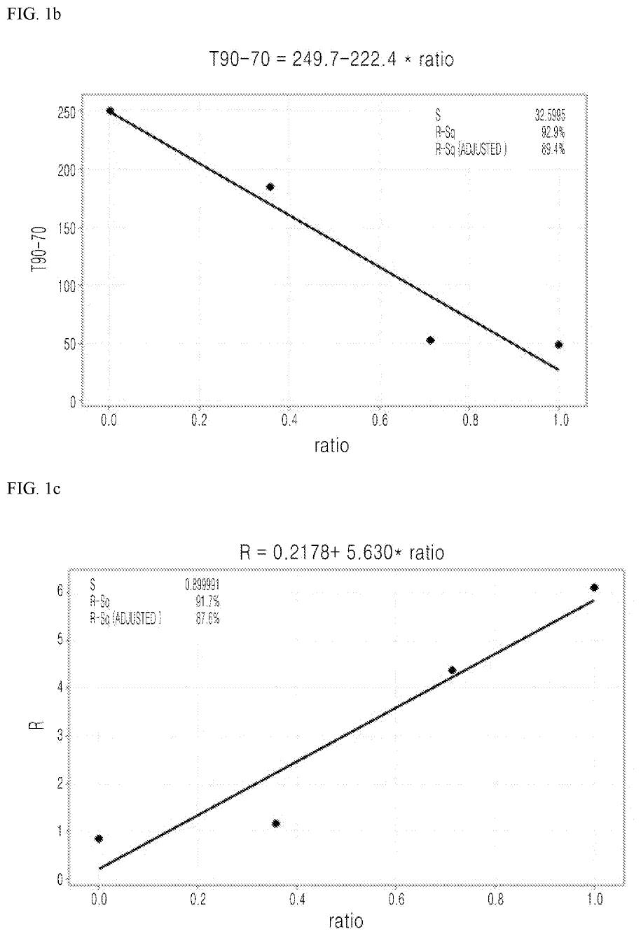

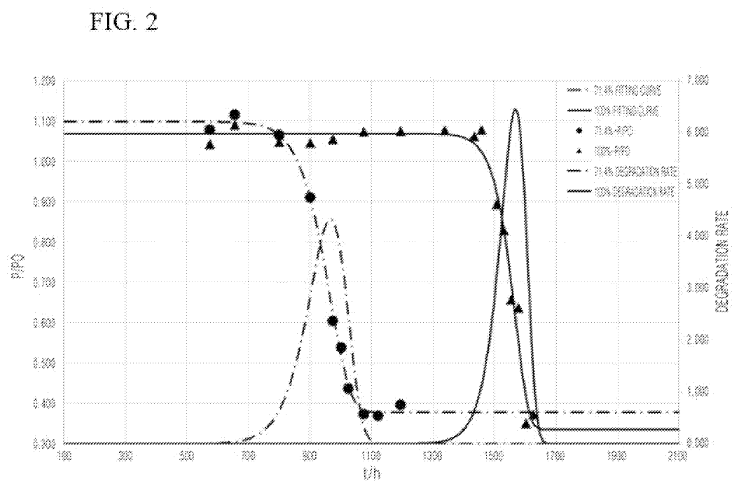

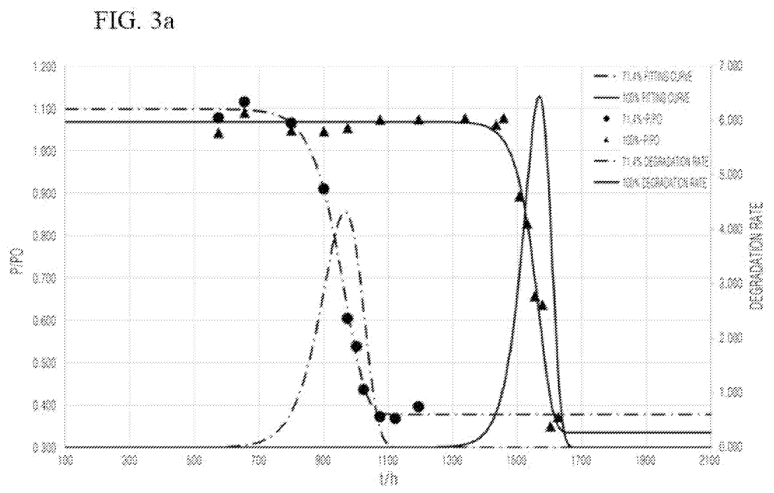

One embodiment of the present disclosure will be described with reference to FIGS. 1a, 1b, 1c, and 2.

First, using a plurality of standard samples with a known recycled material ratio and each having different contents, the relationship between degradation time and/or peak degradation rate and recycled material ratio is obtained.

In this embodiment, a polypropylene (PP) material, PP-GF30, is used as an example for explanation, but the polymer material of the present disclosure is not limited to PP-GF30 and may be other polypropylene materials such as PP-GF40 or other polymer materials other than polypropylene.

### 1-1. Preparation of Standard Samples

Four PP-GF30 standard samples are prepared as shown in the following table:

| Sample | | Recycled | Primary | |
|---|---|---|---|---|
| Number | Material | Material Ratio | Antioxidant Ratio | Secondary Antioxidant Ratio |
| 1 | PP-GF30 | 0% | 150° C.@1000 hrs | Sufficient (present until end of |
| | | | | aging) |
| 2 | PP-GF30 | 35.7% | 150° C.@1000 hrs | Sufficient (present until end of |
| | | | | aging) |
| 3 | PP-GF30 | 71.4% | 150° C.@1000 hrs | Sufficient (present until end of |
| | | | | aging) |
| 4 | PP-GF30 | 100% | 150° C.@1000 hrs | Sufficient (present until end of |
| | | | | aging) |

“150° C.@1000 hrs” in the table indicates that the primary antioxidant ratio is sufficient for performing thermal oxidative aging at 150° C. for 1000 hours.

### 1-2. Thermal Oxidative Aging

(1) The four standard samples described above are each placed in a thermal oven (with air circulation frequency greater than 15 times/hour).
(2) The oven condition is set to 150° C. (temperature varies depending on the type of material).
(3) When aging has been performed for 0 h, 500 h, 800 h, 1150 h, 1350 h, 1800 h, 2100 h, 2500 h, the tensile strength of the four standard samples is measured respectively. Because the degradation onset time of materials is different, there may be some differences in sampling times. For the measurement method for tensile strength, a known method may be adopted. For example, using a universal testing machine, measurements can be made at a tensile speed of 50 mm/min under standard laboratory conditions. The unit of tensile strength is Mpa.

### 1-3. Degradation Curve Fitting

For each of the four standard samples, the following is performed:
(1) Arranging the tensile strength data in chronological order;
(2) Fitting an aging curve through a modified Weibull survival function;
(3) Calculating a derivative of the aging curve to find the peak degradation rate (R) and the degradation time (T90−70) from P/P0=0.9 to P/P0=0.7.

Here, P/P0 represents the physical property retention, which refers to the ratio of some physical property (e.g., density, tensile strength, etc.) after a change or treatment to before the change or treatment when the material undergoes a specific change or treatment. In this embodiment, it refers to the ratio of tensile strength after thermal oxidative aging treatment to tensile strength before thermal oxidative aging treatment.

In this embodiment, P/P0=0.9 indicates that the ratio of tensile strength after thermal oxidative aging treatment to tensile strength before thermal oxidative aging treatment is 0.9 (i.e., 90%), P/P0=0.7 indicates that the ratio of tensile strength after thermal oxidative aging treatment to tensile strength before thermal oxidative aging treatment is 0.7 (i.e., 70%), and the degradation time (T90−70) refers to the time required from P/P0=0.9 to P/P0=0.7.

In this embodiment, the degradation rate is obtained by differentiating the property retention rate (the unit is %) with respect to time, and the peak degradation rate (R) is the maximum value of the degradation rate. Regarding the degradation time (T90−70) and the peak degradation rate (R), these are the same in other embodiments.

FIG. 1a is a diagram showing the change of tensile strength retention rate over time and the change of degradation rate over time during the thermal oxidative aging process of four standard samples. As can be seen from FIG. 1a, the degradation time (T90−70) of standard sample 1 with 0% recycled material ratio is 251 h, and the peak degradation rate (R) is 0.856, the degradation time (T90−70) of standard sample 2 with 35.7% recycled material ratio is 185 h, and the peak degradation rate (R) is 1.171, the degradation time (T90−70) of standard sample 3 with 71.4% recycled material ratio is 53 h, and the peak degradation rate (R) is 4.382, and the degradation time (T90−70) of standard sample 4 with 100% recycled material ratio is 49 h, and the peak degradation rate (R) is 6.122 (as shown in the following table).

| Sample | Recycled | Degradation Time | Peak Degradation |
|---|---|---|---|
| Number | Material Ratio | (T90-70) | Rate (R) |
| 1 | 0% | 251 | 0.856 |
| 2 | 35.7% | 185 | 1.171 |
| 3 | 71.4% | 53 | 4.382 |
| 4 | 100% | 49 | 6.122 |

In the table, the unit of degradation time (T90−70) is hours (h), and the unit of peak degradation rate (R) is (1000 hours)⁻¹((1000 h)⁻¹).

### 1-4. Fitting the Relationship Between Degradation Time/Degradation Rate and Recycled Material Ratio

Fitting the relationship between the degradation time (T90−70) and the recycled material ratio of the four standard samples, the following relationship between the degradation time (T90−70) and the recycled material ratio is obtained:

$$T 90 - 70 = 2 49.7 - 222.4 * ratio .$$

FIG. 1b is a fitting diagram showing the relationship between the degradation time (T90−70) and the recycled material ratio in this embodiment.

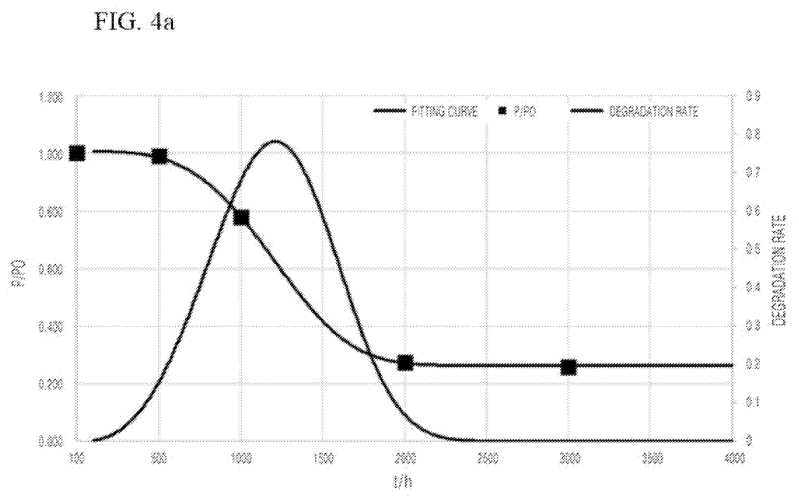

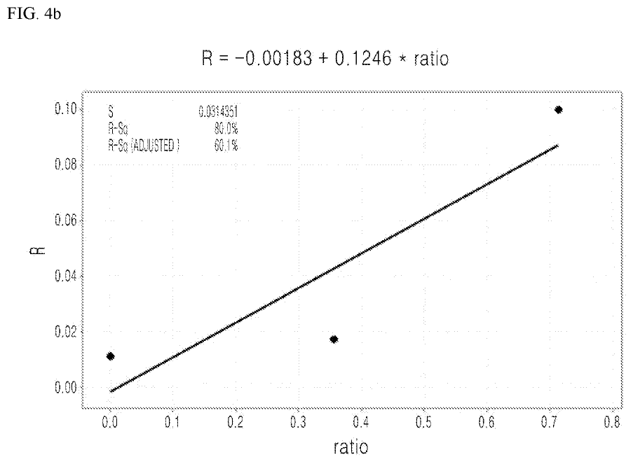

R-Sq and R-Sq (adjusted) in FIG. 1b are indicators used to evaluate the model fit. The closer they are to 1 (100%), the better the fit, and approximately 90% indicates a very high fit (the same applies to FIGS. 1c and 4b).

In this embodiment, the relationship between the degradation time (T90−70) and the recycled material ratio is not limited to the aforementioned relationship (i.e., the relationship shown in FIG. 1b). Depending on differences in sample composition, aging conditions, etc., for example, it may be T90−70=P3+P4*ratio, where 174.8≤P3≤324.6 and −289.1≤P4≤−155.7.

In addition, fitting the relationship between the peak degradation rate (R) and the recycled material ratio of the four standard samples, the following relationship between the peak degradation rate (R) and the recycled material ratio is obtained:

$$R = 0.2178 + 5.63 * ratio .$$

FIG. 1c is a fitting diagram showing the relationship between the peak degradation rate (R) and the recycled material ratio in this embodiment.

In this embodiment, the relationship between the peak degradation rate (R) and the recycled material ratio is not limited to the aforementioned relationship (i.e., the relationship shown in FIG. 1c). Depending on differences in sample composition, aging conditions, etc., for example, it may be R=P1+P2*ratio, where 0.1525≤P1≤0.2831 and 3.941≤P2≤7.319.

After obtaining the relationship between the degradation time (T90−70)/peak degradation rate (R) and the recycled material ratio of PP-GF30 through the above steps, the recycled material ratio of any PP-GF30 sample with unknown recycled material ratio can be calculated; specifically as below.

### 2-1. Sample Reception

Two PP-GF30 samples for analysis are received (recycled material ratio is unknown, sample numbers are 5 and 6, respectively).

### 2-2. Antioxidant Analysis

The ratio of primary antioxidants and secondary antioxidants in the two samples for analysis is analyzed to ensure that the ratio of secondary antioxidants is sufficient during the aging process (if necessary, secondary antioxidants are added until sufficient).

### 2-3. Thermal Oxidative Aging

(1) The two samples for analysis described above are each placed in a thermal oven (with air circulation frequency greater than 15 times/hour).
(2) The oven condition is set to 150° C. (temperature varies depending on the type of material).
(3) When aging has been performed for 0 h, 250 h, 500 h, 800 h, 1000 h, 1800 h, the tensile strength of the samples for analysis is measured respectively. Because the degradation onset time of materials is different, there may be some differences in sampling times. The measurement method for tensile strength is the same as the measurement method described above.

### 2-4. Degradation Curve Fitting

For each of the two samples for analysis, the following is performed:
(1) Arranging the tensile strength data in chronological order;
(2) Fitting an aging curve through a modified Weibull survival function;
(3) Calculating the derivative of the aging curve to find the peak degradation rate (R) and the degradation time (T90−70) from P/P0=0.9 to P/P0=0.7.

FIG. 2 is a diagram showing the change of tensile strength retention rate over time and the change of degradation rate over time during the thermal oxidative aging process of two samples for analysis. As can be seen from FIG. 2, the degradation time (T90−70) of the two samples for analysis is 54 h and 40 h, respectively, and the peak degradation rate (R) is 4.313 and 6.431, respectively.

### 2-5. Calculation of Recycled Material Ratio

For the two samples for analysis described above, according to the relationship between the degradation time (T90−70) and the recycled material ratio T90−70=249.7−222.4*ratio obtained above, and the degradation time (T90−70) obtained here, it can be calculated that the recycled material ratio is 88.0% and 94.3%, respectively.

For the two samples for analysis described above, according to the relationship between the peak degradation rate (R) and the recycled material ratio R=0.2178+5.630*ratio obtained above, and the peak degradation rate (R) obtained here, it can be calculated that the recycled material ratio in the samples for analysis is 72.7% and 110.0%, respectively.

As can be seen above, the recycled material ratio calculated according to the relationship equation of degradation time, and the recycled material ratio calculated according to the relationship equation of degradation rate, are numerically different. However, both methods have errors in calculation, and which method calculates more accurate results depends on sample composition, aging conditions, and the like. In practical operation, the average value of the recycled material ratio calculated by the two methods can be taken, which may be closer to the actual recycled material ratio. For the two samples for analysis mentioned above, the average value of the recycled material ratio calculated by the two methods is 80.4% and 102.2%, respectively.

As can be seen from the above, the degradation time (T90−70), peak degradation rate (R), and recycled material ratio of the two samples for analysis in this embodiment are as shown in the following table.

| | Sample Number | | | | | |
|---|---|---|---|---|---|---|
| | 5 | | | 6 | | |
| | Recycled Material Ratio | | | | | |
| | T90-70 | R | Average | T90-70 | R | Average |
| | Method | Method | Value | Method | Method | Value |
| | 88.0% | 72.7% | 80.4% | 94.3% | 110.0% | 102.2% |
| Degradation Time | 54 | | | 40 | | |
| T90-70 | | | | | | |
| Peak Degradation | 4.313 | | | 6.431 | | |
| Rate (R) | | | | | | |

### Another Embodiment of the Present Disclosure

Even without obtaining the relationship between degradation time or peak degradation rate and recycled material ratio from multiple standard samples, it is possible to determine whether a sample for analysis contains recycled material (whether it is virgin material) through the degradation time and peak degradation rate of the sample for analysis. This will be explained in detail below.

### 1. Sample Reception

Two PP-GF40 samples for analysis are received (company A virgin material and company B virgin material; sample numbers are 7 and 8 respectively; recycled material ratio is 0%).

### 2. Thermal Oxidative Aging

(1) The two samples for analysis are each placed in a thermal oven (with air circulation frequency greater than 15 times/hour).
(2) The oven condition is set to 150° C. (temperature varies depending on the type of material).
(3) When aging has been performed for 0 h, 500 h, 1000 h, 2000 h, 3000 h, 4000 h, the tensile strength of the two samples for analysis is measured respectively. Because the degradation onset time of materials is different, there may be some differences in sampling times. The measurement method for tensile strength is the same as the measurement method described above.

### 3. Degradation Curve Fitting

For each of the two samples for analysis, the following is performed:
(1) Arranging the tensile strength data in chronological order;
(2) Fitting an aging curve through a modified Weibull survival function;
(3) Calculating the derivative of the aging curve to find the peak degradation rate (R) and the degradation time (T90−70) from P/P0=0.9 to P/P0=0.7.

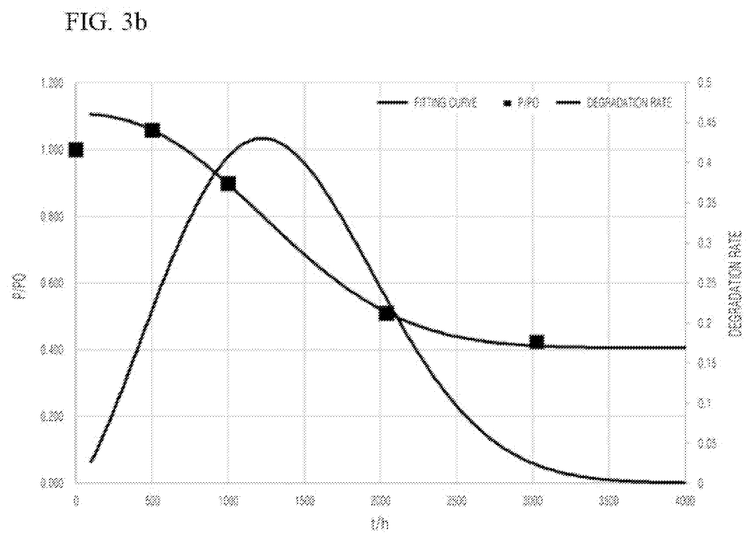

FIG. 3a is a diagram showing the change of tensile strength retention rate overtime and the change of degradation rate over time during the thermal oxidative aging process of company A virgin material (sample number 7), and FIG. 3b is a diagram showing the change of tensile strength retention rate over time and the change of degradation rate over time during the thermal oxidative aging process of company B virgin material (sample number 8). As can be seen from FIG. 3a, the degradation time (T90−70) of the sample for analysis is 474 hours, and the peak degradation rate (R) is 0.430. As can be seen from FIG. 3b, the degradation time (T90−70) of the sample for analysis is 322 hours, and the peak degradation rate (R) is 0.780.

As can be seen from the above, the degradation time (T90−70), peak degradation rate (R), and recycled material ratio of the two samples for analysis in this embodiment are as shown in the following table.

Sample Number

| | 7 | 8 |
|---|---|---|
| Recycled Material Ratio | 0% | 0% |
| Degradation Time (T90-70) | 474 | 322 |
| Peak Degradation Rate (R) | 0.430 | 0.780 |

### 4. Rules for Determining Whether a Sample for Analysis Contains Recycled Material

The inventors of the present disclosure have discovered the following rule through thermal oxidative aging of virgin materials of various materials: if the peak degradation rate (R) is less than 1.0 (unit is (1000 h)⁻¹) and the degradation time (T90−70) is greater than 200 hours, it is typically virgin material (0% recycled material ratio).

In this embodiment, as described above, the peak degradation rate (R) and degradation time (T90−70) of the two virgin material samples for analysis meet the abovementioned rule.

Therefore, based on the peak degradation rate (R) and degradation time (T90−70) of a sample for analysis (not limited to PP-GF40 in this embodiment; it may also be PP-GF30 or other materials) meeting the abovementioned rule, it can be determined that the sample for analysis is a virgin material (0% recycled material ratio).

### Still Another Embodiment of the Present Disclosure

In the above embodiments, the aging curve was fitted through the tensile strength at different times after aging of the sample for analysis. In this embodiment, the aging curve is fitted through the thermogravimetric analysis (TGA) degradation onset temperature after aging of the sample for analysis; specifically, as below. 1. Preparation of Standard Samples

Three PP-GF30 standard samples are prepared as shown in the following table, which are the same as standard samples 1-3 in the first embodiment above.

| Sample | | Recycled | Primary | |
|---|---|---|---|---|
| Number | Material | Material Ratio | Antioxidant Ratio | Secondary Antioxidant Ratio |
| 1 | PP-GF30 | 0% | 150° C.@1000 hrs | Sufficient (present until end of |
| | | | | aging) |
| 2 | PP-GF30 | 35.7% | 150° C.@1000 hrs | Sufficient (present until end of |
| | | | | aging) |
| 3 | PP-GF30 | 71.4% | 150° C.@1000 hrs | Sufficient (present until end of |
| | | | | aging) |

### 2. Thermal Oxidative Aging

(1) The three standard sample materials mentioned above are each placed in a thermal oven (with air circulation frequency greater than 15 times/hour).
(2) The oven condition is set to 150° C. (temperature varies depending on the type of material).
(3) When aging has been performed for 0 h, 500 h, 800 h, 1150 h, 1350 h, 1800 h, 2100 h, 2500 h, the degradation behavior of the three samples for analysis in a nitrogen gas atmosphere is measured through thermogravimetric analysis (TGA), and the degradation onset temperature (thermal decomposition onset temperature) is selected. Because the degradation onset time of materials is different, there may be some differences in sampling times.

### 3. Degradation Curve Fitting

For each of the three standard samples, the following is performed:
(1) Plotting the TGA degradation onset temperature of the material under different thermal oxidative aging times;
(2) Fitting an aging curve through a modified Weibull survival function;
(3) Calculating the derivative of the aging curve to find the peak degradation rate (R).

FIG. 4a is a diagram showing the change overtime of the decrease in TGA thermal decomposition onset temperature compared to the initial sample during the thermal oxidative aging process of three standard samples, and the change of degradation rate over time. In FIG. 4a, δTs refers to the difference between the thermal decomposition onset temperature in TGA analysis of samples with different aging times and the degradation onset temperature of the unaged sample.

As can be seen from FIG. 4a, the peak degradation rate (R) of standard sample 1 with 0% recycled material ratio is 0.011, the peak degradation rate (R) of standard sample 2 with 35.7% recycled material ratio is 0.017, and the peak degradation rate (R) of standard sample 3 with 71.4% recycled material ratio is 0.103 (as shown in the following table).

| Sample | | |
|---|---|---|
| Number | Recycled Material Ratio | Peak Degradation Rate (R) |
| 1 | 0% | 0.011 |
| 2 | 35.7% | 0.017 |
| 3 | 71.4% | 0.103 |

In the table, the unit of peak degradation rate (R) is degrees Celsius/hour (° C./h), representing the degradation onset temperature difference per hour.

### 4. Fitting the Relationship Between Degradation Rate and Recycled Material Ratio

Fitting the relationship between the peak degradation rate (R) and the recycled material ratio, the following relationship between the peak degradation rate (R) and the recycled material ratio is obtained:

$$R = - 0.00183 + 0.1246 * ratio .$$

FIG. 4b is a fitting diagram showing the relationship between the peak degradation rate (R) and the recycled material ratio in this embodiment.

In this embodiment, the relationship between the peak degradation rate (R) and the recycled material ratio is not limited to the relationship (i.e., the relationship shown in FIG. 4b). Depending on differences in sample composition, aging conditions, etc., for example, it may be R=T1+T2*ratio, where −0.00238<T1≤−0.00128 and 0.08722<T2<0.1620.

After obtaining the relationship between the peak degradation rate (R) and the recycled material ratio of PP-GF30 through the above steps, for any PP-GF30 sample with unknown recycled material ratio, as long as the peak degradation rate (R) is obtained in the same way, the recycled material ratio can be calculated according to the relationship between the peak degradation rate (R) and the recycled material ratio. Detailed explanation is omitted here.

### OTHER EMBODIMENTS

In addition to fitting the aging curve through tensile strength and TGA degradation onset temperature as described above, the aging curve can also be fitted through other characteristic properties. For example, the aging curve can be fitted through the carbonyl group index. This method is similar to the methods described above, and detailed explanation is omitted here. The steps after fitting the aging curve are also similar to the methods described above, and detailed explanation is omitted here.

As described above, by adopting the present disclosure, a method capable of performing quantitative analysis of recycled material ratio in polymer materials while excluding the influence of antioxidants may be provided, and a method capable of simply determining whether a polymer material contains recycled material may be provided.

Finally, the above embodiments are only for explaining the technical solutions of this application, not for limiting them. Although the present disclosure has been described in detail with reference to the above embodiments, those skilled in the art can still modify the technical solutions described in the above embodiments, or replace some technical features with equivalents, but such modifications and replacements should ensure that the essence of the corresponding technical solutions does not deviate from the spirit and scope of the technical solutions of the embodiments of the present disclosure.

What is claimed is:

1. A method for calculating a recycled material ratio in a polymer material based on accelerated thermal oxidative aging, the method comprising:
preparing a plurality of standard samples of the polymer material, wherein the plurality of standard samples have a different recycled material ratio and contain primary antioxidants and secondary antioxidants, performing thermal oxidative aging on the plurality of standard samples at a specified temperature, and measuring data of a specified characteristic property of each of the plurality of standard samples at a plurality of times from a start of thermal oxidative aging;
fitting an aging curve for each of the plurality of standard samples according to the data of the specified characteristic property measured during the thermal oxidative aging, and obtaining at least one of a peak degradation rate (R) or a degradation time (T90−70) from P/P0=0.9 to P/P0=0.7 from the aging curve, wherein P/P0 represents a ratio of a value of the specified characteristic property during the thermal oxidative aging to a value of the specified characteristic property before the start of the thermal oxidative aging;
according to at least one of the peak degradation rate (R) or the degradation time (T90−70) of each of the plurality of standard samples, and the recycled material ratio, obtaining at least one of a relationship between the peak degradation rate (R) and the recycled material ratio or a relationship between the degradation time (T90−70) and the recycled material ratio through fitting;
preparing a sample of the polymer material for analysis, and in a state in which the sample for analysis contains primary antioxidants and secondary antioxidants, performing thermal oxidative aging on the sample for analysis under identical conditions to the thermal oxidative aging of the plurality of standard samples, and measuring data of the specified characteristic property;
fitting an aging curve for the sample for analysis according to the data of the specified characteristic property measured during the thermal oxidative aging, and obtaining at least one of the peak degradation rate (R) or the degradation time (T90−70) from P/P0=0.9 to P/P0=0.7 of the sample for analysis from the aging curve; and
calculating the recycled material ratio in the sample for analysis according to at least one of:
the obtained relationship between the peak degradation rate (R) and the recycled material ratio, and the obtained peak degradation rate (R) of the sample for analysis; or
the obtained relationship between the degradation time (T90−70) and the recycled material ratio, and the obtained degradation time (T90−70) of the sample for analysis.

2. The method according to claim 1, wherein the calculating the recycled material ratio in the sample for analysis comprises,
in a situation in which the recycled material ratio in the sample for analysis is calculated according to the relationship between the peak degradation rate (R) and the recycled material ratio and the recycled material ratio in the sample for analysis is calculated according to the relationship between the degradation time (T90−70) and the recycled material ratio, selecting an average value of the two calculated recycled material ratios as the recycled material ratio in the sample for analysis.

3. The method according to claim 1, wherein the fitting an aging curve for each of the plurality of standard samples comprises,
for each of the plurality of standard samples, arranging the data of the specified characteristic property in chronological order, fitting the aging curve through a modified Weibull survival function, calculating a derivative of the aging curve, and obtaining at least one of the peak degradation rate (R) or the degradation time (T90−70) from the derivative of the aging curve.

4. The method according to claim 1, wherein the fitting an aging curve for the sample for analysis comprises,
for the sample for analysis, arranging the data of the specified characteristic property in chronological order, fitting the aging curve through a modified Weibull survival function, calculating a derivative of the aging curve, and obtaining at least one of the peak degradation rate (R) or the degradation time (T90−70) from the derivative of the aging curve.

5. The method according to claim 1, wherein
the polymer material is PP-GF30,
the specified characteristic property is tensile strength, and
the obtained relationship between the peak degradation rate (R) and the recycled material ratio is R=P1+P2*ratio, wherein a unit of the peak degradation rate (R) is (1000 hours)⁻¹, 0.1525≤P1≤0.2831, and 3.941≤P2≤7.319.

6. The method according to claim 5, wherein P1=0.2178 and P2=5.630.

7. The method according to claim 1, wherein
the polymer material is PP-GF30,
the specified characteristic property is tensile strength, and
the obtained relationship between the degradation time (T90−70) and the recycled material ratio is T90−70=P3+P4*ratio, wherein a unit of the degradation time (T90−70) is hours, 174.8≤P3≤324.6, and −289.1≤P4≤−155.7.

8. The method according to claim 7, wherein P3=249.7 and P4=−222.4.

9. The method according to claim 1, wherein
the polymer material is PP-GF30,
the specified characteristic property is TGA degradation onset temperature, and
the obtained relationship between the peak degradation rate (R) and the recycled material ratio is R=T1+T2*ratio, wherein a unit of the peak degradation rate (R) is degrees Celsius/hour, −0.00238<T1≤−0.00128, and 0.08722<T2<0.1620.

10. The method according to claim 9, wherein T1=−0.00183 and T2=0.1246.

11. The method according to claim 1, wherein the preparing a plurality of standard samples comprises providing the primary antioxidants in an amount sufficient to perform 1000 hours of thermal oxidative aging under the specified temperature, and providing the secondary antioxidants in an amount sufficient to remain present until an end of thermal oxidative aging.

12. The method according to claim 1, wherein the preparing a sample of the polymer material for analysis comprises, before performing thermal oxidative aging on the sample for analysis, analyzing a ratio of primary antioxidants and secondary antioxidants in the sample for analysis to ensure that an amount of the secondary antioxidants is sufficient during the thermal oxidative aging, and based on the amount of the secondary antioxidants being insufficient, adding secondary antioxidants until sufficient.

13. A method for determining whether a polymer material contains a recycled material based on accelerated thermal oxidative aging, the method comprising:
preparing a sample of a polymer material for analysis, and in a state in which the sample for analysis contains primary antioxidants and secondary antioxidants, performing thermal oxidative aging on the sample for analysis at a specified temperature, and measuring data of a specified characteristic property of the sample for analysis at a plurality of times from a start of thermal oxidative aging;
fitting an aging curve for the sample for analysis according to the data of the specified characteristic property measured during the thermal oxidative aging, and obtaining a peak degradation rate (R) and a degradation time (T90−70) from P/P0=0.9 to P/P0=0.7 of the sample for analysis from the aging curve, wherein P/P0 represents a ratio of a value of the specified characteristic property during the thermal oxidative aging to a value of the specified characteristic property before the start of thermal oxidative aging; and
determining that the sample for analysis is a virgin material not containing recycled material based on the peak degradation rate (R) being less than a first threshold and the degradation time (T90−70) being greater than a second threshold.

14. The method according to claim 13, wherein the determining comprises
determining that the sample for analysis contains recycled material based on the peak degradation rate (R) being greater than or equal to the first threshold or the degradation time (T90−70) being less than or equal to the second threshold.

15. The method according to claim 13, wherein the fitting an aging curve for the sample for analysis comprises
arranging the data of the specified characteristic property in chronological order, fitting the aging curve through a modified Weibull survival function, calculating a derivative of the aging curve, and obtaining the peak degradation rate (R) and the degradation time (T90−70) from the derivative of the aging curve.

16. The method according to claim 13, wherein
the polymer material is PP-GF40 or PP-GF30,
the specified characteristic property is tensile strength, and
the first threshold is 1.0(1000 hours)⁻¹ and the second threshold is 200 hours.
